(19) United States
(12) Patent Application Publication
(10) Pub. No.: US 2005/0143654 A1
(43) Pub. Date: Jun. 30, 2005

(54) Systems and methods for segmented volume rendering using a programmable graphics pipeline

(76) Inventors: Karel Zuiderveld, Minnetonka, MN (US); Steve Demlow, Roseville, MN (US); Matt Cruikshank, Maple Grove, MN (US)

Correspondence Address: SCHWEGMAN, LUNDBERG, WOESSNER & KLUTH, P.A., P.O. BOX 2938, MINNEAPOLIS, MN, 55402-0938, US

(21) Appl. No.: 10/996,343

(22) Filed: Nov. 23, 2004

Related U.S. Application Data
(60) Provisional application No. 60/525,791, filed on Nov. 29, 2003.

(51) Int. Cl. A61B 8/00

(52) U.S. Cl. 600/443

(57) ABSTRACT

This document describes systems and methods for, among other things, visualizing 3D volumetric data comprising voxels using different segmentation regions. A segmentation mask vector is associated with each voxel, which defines to which segmentation region that voxel belongs. During the visualization, segmentation masks are interpolated to obtain a vector of segmentation mask weights. For each sample point, a vector of visualization values is multiplied by a vector of segmentation mask weights to produce a composite fragment value. The fragment values are combined into pixel values using compositing. The systems and methods leverage the computational efficiency of commodity programmable video cards to determine accurately subsampled partial contribution weights of multiple segmented data regions to allow correct per-fragment combination of segment specific characteristics such as color and opacity, which is suitable for many applications, including volume rendering.

This application claims priority to U.S. Provisional Application No. 60/525,791, filed Nov. 29, 2003 which is incorporated herein by reference.

### COPYRIGHT NOTICE

A portion of the disclosure of this document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever. The following notice applies to the software and data as described below and in the drawings that form a part of this document: Copyright 2003, Vital Images, Inc. All Rights Reserved.

### TECHNICAL FIELD

This document pertains generally to computerized systems and methods for processing and displaying three dimensional imaging data, and more particularly, but not by way of limitation, to computerized systems and methods for segmented volume rendering using a programmable graphics pipeline.

### BACKGROUND

Because of the increasingly fast processing power of modern-day computers, users have turned to computers to assist them in the examination and analysis of images of real-world data. For example, within the medical community, radiologists and other professionals who once examined x-rays hung on a light screen now use computers to examine volume data obtained using various technologies. Such technologies include imaging devices such as ultrasound, computed tomography (CT), magnetic resonance (MR), positron emission tomography (PET), single photon emission computed tomography (SPECT), and other such image acquisition technologies. Many more image acquisition techniques, technologies, and devices will likely arise as medical imaging technology evolves.

Each of these imaging procedures uses its particular technology to generate volume data. For example, CT uses an x-ray source that rapidly rotates around a patient. This typically obtains hundreds or thousands of electronically stored pictures of the patient. As another example, MR uses radio-frequency waves to cause hydrogen atoms in the water content of a patient's body to move and release energy, which is then detected and translated into an image. Because each of these techniques records data from inside the body of a patient to obtain and reconstruct data, and because the body is three-dimensional, the resulting data represents a three-dimensional image, or volume. In particular, CT and MR both typically provide three-dimensional (3D) data.

3D representations of imaged structures have typically been produced through the use of techniques such as surface rendering and other geometric-based techniques. Because of known deficiencies of such techniques, volume-rendering techniques have been developed as a more accurate way to render images based on real-world data. Volume rendering is a direct representation of a three-dimensional data set. However, volume rendering typically uses and processes a huge amount of volumetric data. Because of the huge amount of data involved, efficient storage and processing techniques are needed to provide a useful tool for the user.

One technique for processing the large amount of data includes segmenting the data into segmentation regions (also referred to as “segments”) that are of interest to the user. Segmenting data is useful both from a user perspective and a system perspective. From a user perspective, segmenting data narrows the amount of data to be viewed by the user to a subset that is of particular interest to the user. In addition, segmentation can also be used to highlight specific anatomical regions in a dataset, for example, by assigning different coloring schemes or rendering algorithms to individual segments. From a system perspective, data segmentation can reduce the amount of data that undergoes further processing, storage, and display. This increases the system's efficiency, which, in turn, increases the speed at which useful images can be provided to the user. There exist many data segmentation techniques that accommodate various structures of interest in the volumetric data. There is a need to provide volume rendering techniques that efficiently use the segmented data to accurately produce rendered 3D representations of imaged structures.

### BRIEF DESCRIPTION OF THE DRAWINGS

In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

FIG. 1 is a block diagram illustrating generally, among other things, one example of portions of an imaging visualization system, and an environment within which it is used, for processing and displaying volumetric data, such as of a human or animal or other subject or any other imaging region of interest.

FIG. 2 is a schematic illustration of one example of a remote or local user interface.

FIG. 3 is a block diagram illustrating one example of portions of a system that uses one or more fragment programs.

FIG. 4 is a schematic diagram illustrating a conceptual example of a programmable graphics pipeline of a GPU of a video card.

FIG. 5 is a block diagram illustrating generally, among other things, one example of a technique of acquiring, rendering, and visualizing volumetric data.

FIG. 6 is a flow chart illustrating generally an exemplary overview of a technique of volume rendering.

FIG. 7 is a schematic illustration of one conceptualization of volume rendering using ray-casting (although other volume rendering techniques could also be used).

FIG. 8 is a further schematic illustration of the volume rendering conceptualization of FIG. 7, but illustrating at a higher magnification a small portion of a ray as it passes through a neighborhood of eight neighboring voxels (that are defined by their centerpoints).

FIG. 9 is an illustration of one example of using transfer functions to overlay different visual characteristics to voxel intensity data that is associated with different segmentation regions.

FIG. 10 is a schematic diagram illustrating conceptually how, for each sample point, a fragment program uses an interpolated voxel intensity value, an interpolated vector of segmentation weights, and transfer functions.

FIG. 11 is a schematic diagram illustrating conceptually one example of various data structures associated with an exemplary fragment shading segmented volume rendering process.

FIG. 12 is a schematic diagram, corresponding to the neighborhood block of FIG. 8, of a neighborhood block comprising voxel points and a sample point contained within that neighborhood block.

FIG. 13 is a schematic diagram, corresponding to the same neighborhood block of FIG. 12, but with the voxel points represented by their respective segmentation mask values composed of four channels of 4-bit unsigned integer data values.

FIG. 14 is a schematic diagram illustrating a result of a trilinear interpolation (on a component-by-component basis) on a sample point having parametric (x, y, z) coordinates.

### DETAILED DESCRIPTION

In the following detailed description, reference is made to the accompanying drawings which form a part hereof, and in which is shown by way of illustration specific embodiments in which the invention may be practiced. These embodiments, which are also referred to herein as “examples,” are described in sufficient detail to enable those skilled in the art to practice the invention, and it is to be understood that the embodiments may be combined, or that other embodiments may be utilized and that structural, logical and electrical changes may be made without departing from the scope of the present invention. The following detailed description is, therefore, not to be taken in a limiting sense, and the scope of the present invention is defined by the appended claims and their equivalents.

In this document, the terms “a” or “an” are used, as is common in patent documents, to include one or more than one. In this document, the term “or” is used to refer to a nonexclusive or, unless otherwise indicated. Furthermore, all publications, patents, and patent documents referred to in this document are incorporated by reference herein in their entirety, as though individually incorporated by reference. In the event of inconsistent usages between this documents and those documents so incorporated by reference, the usage in the incorporated reference(s) should be considered supplementary to that of this document; for irreconcilable inconsistencies, the usage in this document controls.

Some portions of the detailed descriptions which follow are presented in terms of algorithms and symbolic representations of operations on data bits within a computer memory. These algorithmic descriptions and representations are the ways used by those skilled in the data processing arts to most effectively convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like. It should be borne in mind, however, that all of these and similar terms are to be associated with the appropriate physical quantities and are merely convenient labels applied to these quantities. Unless specifically stated otherwise as apparent from the following discussions, terms such as “processing” or “computing” or “calculating” or “determining” or “displaying ” or the like, refer to the action and processes of a computer system, or similar computing device, that manipulates and transforms data represented as physical (e.g., electronic) quantities within the computer system's registers and memories into other data similarly represented as physical quantities within the computer system memories or registers or other such information storage, transmission or display devices.

System Environment Overview

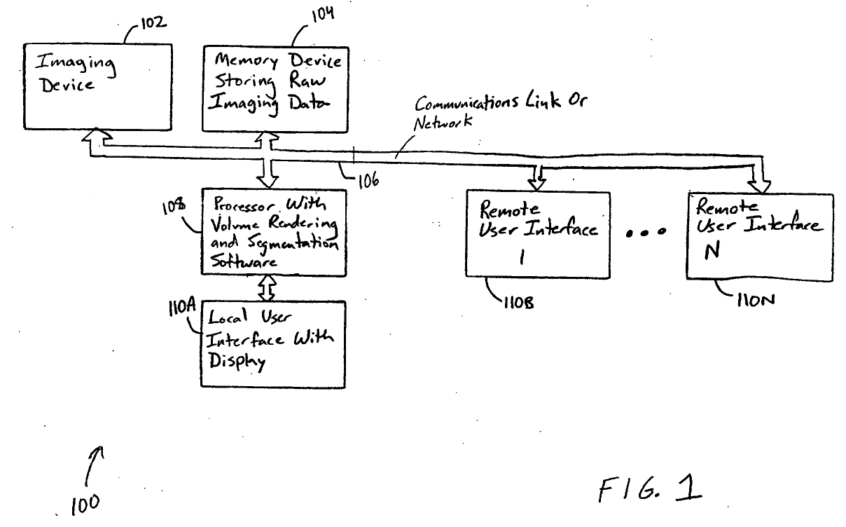

FIG. 1 is a block diagram illustrating generally, among other things, one example of portions of an imaging visualization system 100, and an environment within which it is used, for processing and displaying volumetric data of a human or animal or other subject or any other imaging region of interest. In this example, the system 100 includes (or interfaces with) an imaging device 102. Examples of the imaging device 102 include, without limitation, a computed tomography (CT) scanner or a like radiological device, a magnetic resonance (MR) imaging scanner, an ultrasound imaging device, a positron emission tomography (PET) imaging device, a single photon emission computed tomography (SPECT) imaging device, and other image acquisition modalities. Many more imaging techniques and devices will likely arise as medical imaging technology evolves. Such imaging techniques may employ a contrast agent to enhance visualization of portions of the image (for example, a contrast agent that is injected into blood carried by blood vessels) with respect to other portions of the image (for example, tissue, which typically does not include such a contrast agent).

The imaging device 102 outputs volumetric (3 dimensional) imaging data. The 3D imaging data is provided as a rectilinear array of volume elements called voxels. Each voxel has an associated intensity value, referred to as a gray value. The different intensity values provide imaging information. For example, for CT images, the different intensity values represent the different densities of the underlying structures being imaged. For example, bone voxel values typically exceed 600 Hounsfield units, tissue voxel values are typically less than 100 Hounsfield units, and contrast-enhanced blood vessel voxel values fall somewhere between that of tissue and bone.

Hardware Environment Overview

In the example of FIG. 1, the system 100 also includes zero or more computerized memory devices 104, which are operatively coupled to the imaging device 102, such as by at least one local and/or wide area computer network or other communications link 106. A memory device 104 stores volumetric data that it receives from the imaging device 102. Many different types of memory devices 104 will be suitable for storing the volumetric data. A large volume of data may be involved, particularly if the memory device 104 is to store data from different imaging sessions and/or different patients.

In this example, one or more computer processors 108 are coupled to the memory device 104 through the communications link 106 or otherwise. The processor 108 is capable of accessing the volumetric data that is stored in the memory device 104. The processor 108 executes a segmentation algorithm that classifies each of the individual voxels from the volumetric dataset into identifies imaging data voxels pertaining to one or more segments of interest. The term “segmenting” refers to separating the volumetric data associated with a particular property from other volumetric data. In one illustrative example, but not by way of limitation, the data segmentation algorithm identifies and labels voxels associated with vessels or other tubular structures. Then, segmented volume rendering creates a visual depiction using the voxels that were segmented into one or more segmentation regions. The visual depiction is displayed, such as on a computer monitor screen or other two-dimensional planar display.

In one example, the system 100 optionally includes one or more local user interfaces 110A, which are locally coupled to the processor 108, and/or optionally includes one or more remote user interfaces 110B-N, which are remotely coupled to the processor 108, such as by using the communications link 106. Thus, in one example, the user interface 110A and the processor 108 form an integrated imaging visualization system 100. In another example, the imaging visualization system 100 implements a client-server architecture with the processor(s) 108 acting as a server for processing the volumetric data for visualization, and communicating graphic display data over the at least one communications link 106 for display on one or more of the remote user interfaces 110B-N. In either example, the user interface 110 includes one or more user input devices (such as a keyboard, mouse, web browser, etc.) for interactively controlling the data segmentation and/or volume rendering being performed by the processor(s) 108 and the graphics data being displayed.

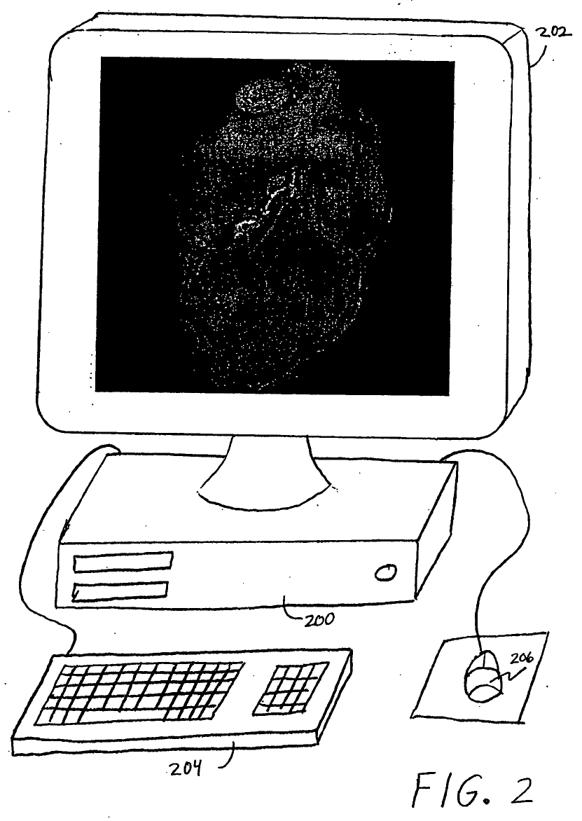

FIG. 2 is a schematic illustration of one example of a remote or local user interface 110. In this example, the user interface 110 includes a personal computer workstation 200 that includes an accompanying monitor display screen 202, keyboard 204, and mouse 206. In an example in which the user interface 110 is a local user interface 110A, the workstation 200 includes the processor 108 for performing data segmentation and volume rendering for data visualization. In another example, in which the user interface 110 is a remote user interface 110B-N, the client workstation 200 includes a processor that communicates over the communications link 106 with a remotely located server processor 108.

Hardware Environment Example

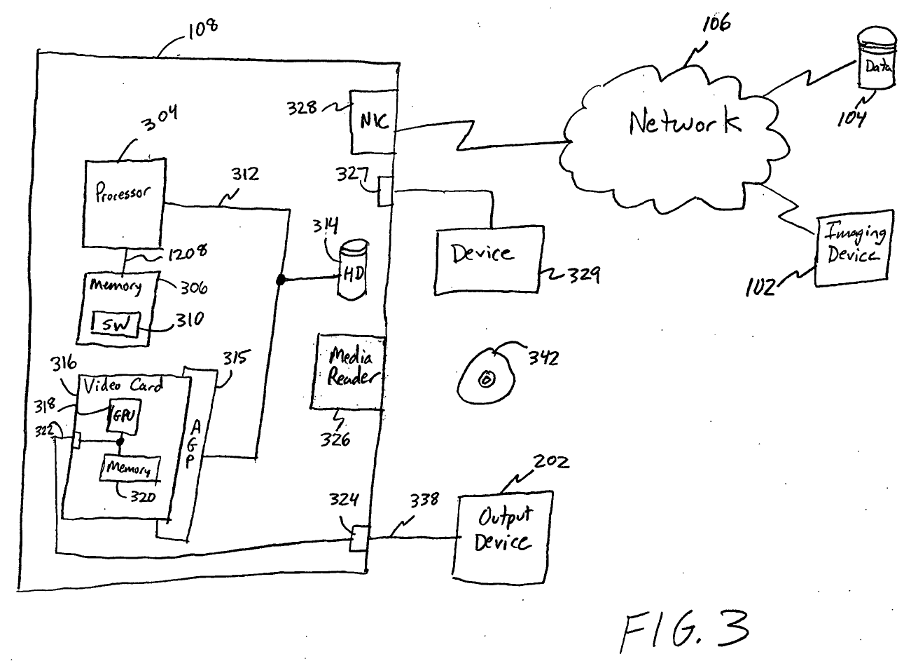

FIG. 3 is a block diagram illustrating one example of portions of a system 300 that uses one or more fragment programs. In this example, the system 300 includes computer 108 having processor 304 and a memory 306 coupled thereto. The processor 304 is operatively coupled to a bus 312. A programmable video card 316 is operatively coupled to the bus 312, such as via a PCI Express (PCI-X) or Advanced Graphic Port (AGP) 315. The video card 316 includes a graphics processing unit (GPU) 318. The. GPU 318 is operatively coupled to a video card memory 320 of the video card 316. The video card 316 is also coupled, at 322, to a video output port 324. The video card output port 324 is also coupled, at 338, to a video output device 202. The video output device 202 includes one or more of a computer monitor, a video recording device, a television, and/or any other device capable of receiving an analog or digital video output signal.

The system 300 includes software 310 operable on the processor 304 to obtain volumetric (3D) data, comprising voxels, such as from one or more of a networked or hardwired medical imaging device 102, a networked data repository 104 (such as a computer database), a computer readable medium 342 readable by a media reader 326 coupled to the bus 312, and/or a hard drive 314 internal or external to the computer 108.

The software 310 is further operable on the processor 304 to execute a segmentation algorithm to classify the 3D data into separate objects of interest. The result of this segmentation algorithm is a segmentation mask that can have an arbitrary number of objects. In the segmentation mask, each voxel is associated with only one object. This process is also referred to herein as segmenting a volume dataset into one or more regions of interest.

In one example, the software 310 sends the volumetric data, the segmentation mask, a multichannel transfer function table, and a fragment program over the bus 312 to the video card 316. The transfer function table includes a separate channel corresponding to each segmentation region.

The fragment program is operable on the video card 316 to process sample points within the volumetric data. Operating the fragment program on the video card 316 also derives segmentation weights using trilinear interpolation for each individual sample point value. The fragment program also multiplies a visualization value from each transfer function channel by its corresponding segmentation weight to obtain a contribution of each transfer function channel to a final composite fragment value. Fragment values are aggregated to form a final composite image output from the video output port 324, such for display, storage, or archiving.

In one example, the computer 108 includes a network interface card (NIC) 328. The NIC 328 may include a readily available 10/100 Ethernet compatible card or a higher speed network card such as a gigabit Ethernet or fiber optic enabled card. Other examples include wireless network cards that operate at one or more transmission speeds, or multiple NICs 328 to increase the speed at which data can be exchanged over a network 106.

In another example, the computer 108 includes at least one port 327. Examples of the port(s) 327, include a Universal Serial Bus (USB) port, an I.E.E.E. 1394 enabled ports, a serial port, an infrared port, audio ports, and/or any other input or output port 327. The port 327 is capable connection with one or more devices 329. Examples of device(s) 329 include a keyboard, a mouse, a camera, a pen computing device, a printer, a speaker, a USB or other connection type enabled network card, a video capture device, a video display device, a storage device, a Personal Digital Assistant (PDA), or the like.

The software 310 may be available to the system 300 from various locations, such as a memory 310, a hard disk 314, a computer readable medium 342, a network 106 location, the internet, or any other such location that a computer 108 executing the software 310 has access to.

Video Card Hardware Example

The video card 316 is capable of producing 3D images. Because the video card 316 is programmable, a fragment (or other) program can be executed on the video card 316 for performing custom processing.

In general, a video card typically operates by drawing geometric primitives in 3D (such as triangles defined by three vertices). A rasterizer projects the geometric primitives into a 2D frame buffer for eventual display. The frame buffer includes a 2D array of pixels. The rasterization determines which pixels are altered by the projected triangle. For example, if the triangle is red, the rasterization turns all of the pixels under the projected triangle red. This causes a red triangle to be displayed on the screen. However, for each pixel that is covered by the projected triangle, the video card can do more calculations to compute the color and other characteristics of those pixels. For instance, instead of just having a red triangle, the triangle may include a red vertex, blue vertex, and a green vertex. The video card is capable of processing the colors of the triangle to provide a smooth blend of colors across the triangle. The triangle can be further processed using texture maps. A texture map essentially pastes a 2D image onto a surface of a triangle. This technique is typical in video games, such as to give walls certain appearances such as brick or stone and also to give characters faces and clothing.

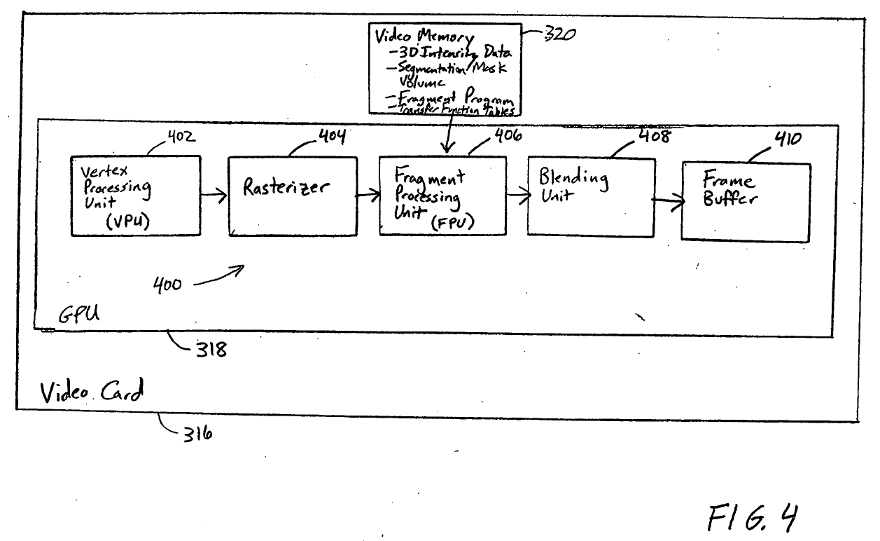

FIG. 4 is a schematic diagram illustrating a conceptual example of a programmable graphics pipeline 400 of the GPU 318 of the video card 316. In this example, the pipeline 400 includes a vertex processing unit (VPU) 402, a rasterizer 404, a fragment processing unit (FPU) 406, a blending unit 408, and a frame buffer 410.

The VPU 402 processes 3D vertices of triangles or like geometric primitives. The VPU 402 typically independently manipulates the vertex positions of these geometric primitives. However, the VPU 402 can also be used manipulate additional attributes or data associated with a vertex, such as 3D texture index coordinates (e.g., (x, y, z) coordinates) for indexing a 3D position of a voxel within a volumetric intensity dataset or a corresponding segmentation mask volume.

The rasterizer 404 receives data output from the VPU 402. The rasterizer 404 rasterizes the geometric primitive to determine which pixels on a display 202 of the output device 202 are contributed to by the geometric primitive. This information is output to the FPU 406. The FPU 406 executes one or more fragment programs, as discussed above. The fragment programs are received from the video memory 320, along with 3D intensity data and segmentation mask volume data and a multichannel transfer function table. The FPU 406 outputs fragments to a blending unit 408. The blending unit 408 combines multiple layers of fragments into a pixel that is stored in the frame buffer 410.

Image Acquisition, Rendering and Visualization Overview

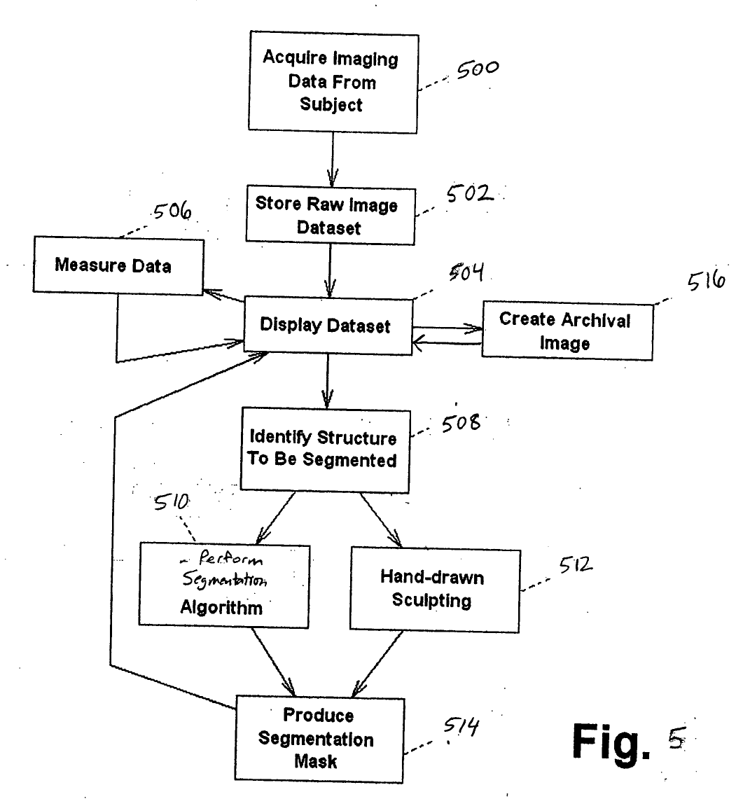

FIG. 5 is a block diagram illustrating generally, among other things, one example of a technique of acquiring, rendering, and visualizing volumetric data. At 500, a volumetric dataset is acquired from a human, animal, or other subject of interest, such as by using one of the imaging modalities discussed above. Alternatively, the volumetric dataset is acquired by accessing previously acquired and stored data. At 502, the volumetric dataset is stored. In one example, this act includes storing in a network-accessible computerized memory device 104. At 504, the volumetric dataset is displayed to a user on a 2D screen as a rendered 3D view. At 516, an archival image of the rendered 3D view is optionally created and stored in a memory device 104, before returning to 504. At 506, one or more aspects of the displayed dataset is optionally measured, before returning to 504. In one example, this includes measuring the diameter of a blood vessel to assess stenosis. In another example, this includes automatically or manually measuring the size of a displayed bone, organ, tumor, etc. At 508, a structure to be segmented from other data is identified, such as by receiving user input. In one example, the act of identifying the structure to be segmented is responsive to a user using the mouse 206 to position a cross-hair or other cursor over a structure of interest, such as a coronary or other blood vessel, as illustrated in FIG. 2. This initiates a segmentation algorithm that is performed at 510, thereby producing a resulting segmentation mask at 514. One example of a data segmentation algorithm is described in Krishnamoorthy et al., U.S. patent application Ser. No. 10/723,445 entitled “SYSTEMS AND METHODS FOR SEGMENTING AND DISPLAYING TUBULAR VESSELS IN VOLUMETRIC IMAGING DATA,” which was filed on Nov. 26, 2003, and which is assigned to Vital Images, Inc., and which is incorporated by reference herein in its entirety, including its description of data segmentation. However, many segmentation algorithms exist, and the present system can also use any other such segmentation algorithm or technique.

At 512, a user performs hand-drawn sculpting, such as by using the mouse 206 to draw an ellipse or curve on the displayed 3D view. This is projected through the volumetric data. A resulting cone or like 3D shape is formed, which can be used to specify a desired segmentation of data inside (or, alternatively, outside) the cone or like 3D shape. This produces a segmentation mask at 514. Segmentation may also involve a combination of hand-drawn sculpting at 512 and performing an automated segmentation algorithm at 510.

After the segmentation mask is produced at 514, the segmented data is redisplayed at 504. In one example, the act of displaying the segmented data at 504 includes displaying the segmented data (e.g., with color highlighting or other emphasis) along with the non-segmented data. In another example, the act of displaying the segmented data at 504 includes displaying only the segmented data (e.g., hiding the non-segmented data). In a further example, whether the segmented data is displayed alone or together with the non-segmented data is a parameter that is user-selectable, such as by using a web browser or other user input device portion of the user interface 110.

After the segmentation mask is produced at 514, the then-current segmentation mask can be archived, such as to a memory device 104. The archived segmentation mask(s) can then later be restored, if desired.

Volume Rendering Overview

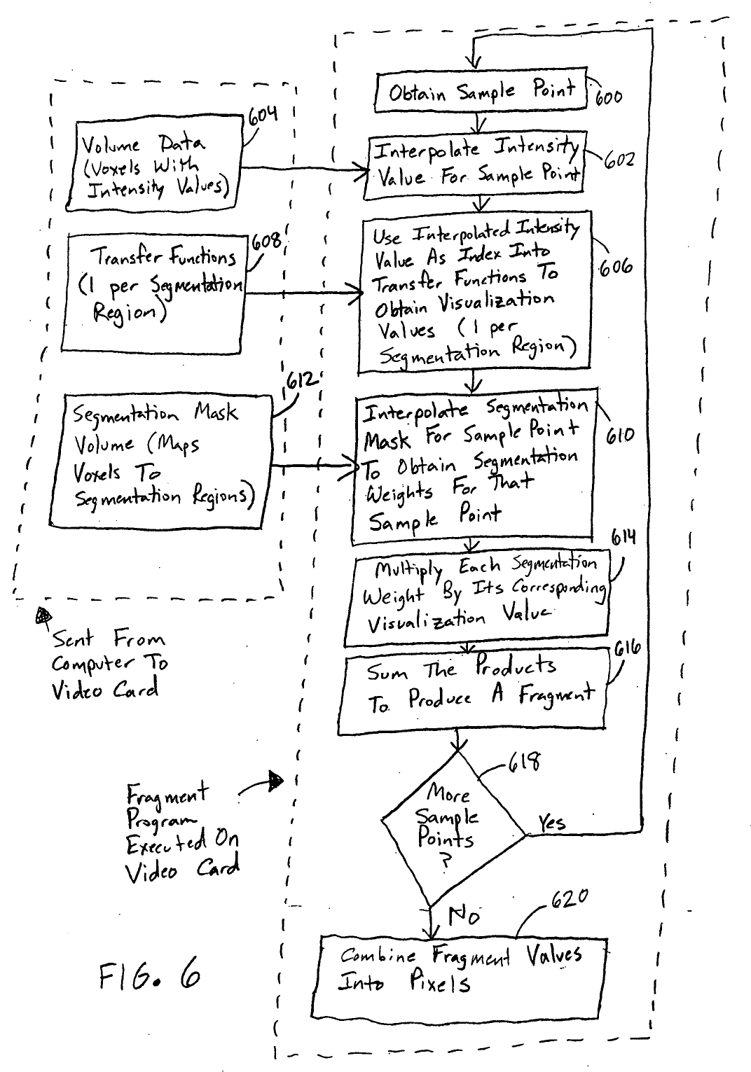

FIG. 6 is a flow chart illustrating generally an exemplary overview of the present technique of volume rendering. This technique uses many sample points taken at various locations within the volume of the 3D imaging data, and transforms these sample points into fragments, which are then combined into pixels that are placed in a frame buffer for display to a user. At 600, one of the sample points is obtained. The sample points typically do not exhibit a one-to-one correspondence with the voxels being sampled. For example, a particular voxel may be sampled by more than one sample point. Moreover, the sample points need not be located exactly at the center point defined by each voxel. Therefore, at 602, an intensity value for the sample point is interpolated (or otherwise computed) using volume data (i.e., voxels with corresponding intensity values) that is received at 604. This assigns an intensity value to each sample point that is determined from neighboring voxels. The intensity values of voxels that are closer to the sample point affect the intensity assigned to the sample point more than the intensity values of voxels that are more distant from the sample point.

At 606, the interpolated intensity value for the sample point is used to calculate the visualization values to be assigned to the sample point. This calculation is performed for each of the segmented regions contained in the segmentation mask. In one example, there is a separate transfer function that is received at 608 for each of the segmented regions. The interpolated intensity value then serves as an index into the individual transfer functions that are received at 608. Therefore, using the intensity value as an index, and with each transfer function contributing a separate RGBA visualization value, a particular sample point obtains a number of RGBA. The number of the RGBA visualization values corresponds to the number of segmentation regions.

At 610, a segmentation mask for the sample point is interpolated (or otherwise computed) using segmentation mask volume data that is received at 612. The segmentation mask volume data includes a segmentation mask vector assigned to each voxel that defines which one of the segmentation regions the voxel was segmented into. Again, because the sample points do not necessarily exhibit a one-to-one correspondence to the voxels, interpolation (or a like filtering or combination technique) is performed. At 610, the interpolation yields segmentation weights for the sample point. The segmentation weights indicate to which degree a particular sample point belongs to the various sample regions (thus, although a voxel belongs to a single segmentation region, a sample point can belong to more than one segmentation region, to varying degrees). The segmentation mask value of voxels that are closer to the sample point affect the segmentation weights assigned to the sample point more than the segmentation mask values of voxels that are more distant from the sample point.

At 614, each segmentation weight is multiplied by the corresponding RGBA visualization value obtained from the corresponding transfer function. At 616, these products are summed to produce an output value for this fragment. The operations at 614 and 616 may be combined into a single “multiply-and-accumulate” operation, as is typically available on a digital signal processing (DSP) oriented processor, and are illustrated separately in FIG. 6 for conceptual clarity. At 618, a check is performed to determine whether more sample points need to be processed. If so, process flow returns to 600. Otherwise, at 620, the fragments are combined into pixels. Such combination may use back-to-front or front-to-back compositing techniques, or any other fragment combination technique known in volume rendering. In FIG. 6, 618 is illustrated as preceding 620 for conceptual clarity. However, in one implementation, intermediate values for each pixel are computed for each sample point, and iteratively updated as further sample points are processed.

FIG. 6 illustrates sending the volumetric data at 604, the segmentation mask volume at 612, the transfer functions at 608, and a fragment program to a programmable computer-video card 316 for executing the fragment program. The programmable video card 316 is programmable via an application programming interface (API). Some examples include video cards 316 that comply with one or more of the various versions of OpenGL developed originally by Silicon Graphics, Inc. In other examples the video card is compliant with Microsoft Corporation's Direct3D standard. Such cards are readily available from manufacturers such as nVidia and ATI.

In one example, the interpolation (e.g., at 602 and 610) uses a command in the fragment program that is native to the video card 316 to cause a trilinear interpolation to occur. For example, in the OpenGL ARB_FRAGMENT_PROGRAM extension a “TEX” command with a “3D” parameter causes the video card to perform a texture lookup with a trilinear interpolation as part of the lookup, assuming that the OpenGL state was previously configured for trilinear interpolation. The output of such a command includes results that are trilinearly interpolated. However, the trilinear interpolation need not be performed using a native video card command. A trilinear interpolation can be included in the code of the fragment program itself. An example of a trilinear interpolation algorithm in pseudo code is as follows:

///////////////////////////////////////////////////////////////////////////////////////////
/////////////////////////////////////////
// Function that does 1D linear interpolation − weight is in [0..1], lowValue and
highValue are
// arbitrary
///////////////////////////////////////////////////////////////////////////////////////////
/////////////////////////////////////////
float lerp(float weight, float lowValue, float highValue)
{
return lowValue + ((highValue − lowValue) * weight);
}
// Sample input for a trilerp operation
int neighborhood[2][2][2]; // 3D neighborhood of int values, indexed by X, then Y,
then Z
float samplePosition[3] = { 0.25, 0.5, 0.41 }; // Positions in each dimension, in [0..1], to
sample at (i.e. the weights of the interpolation in each dimension). These values
correspond to the example parametric position in FIG. 14.
// Trilinear Interpolation implementation:
// Do 4 lerps in the X axis
float x00 = lerp(samplePosition[0], neighborhood[0][0][0], neighborhood[1][0][0]);
float x01 = lerp(samplePosition[0], neighborhood[0][0][1], neighborhood[1][0][1]);
float x10 = lerp(samplePosition[0], neighborhood[0][1][0], neighborhood[1][1][0]);
float x11 = lerp(samplePosition[0], neighborhood[0][1][1], neighborhood[1][1][1]);
// Do 2 lerps in the Y axis
float y00 = lerp(samplePosition[1], x00, x01);
float y01 = lerp(samplePosition[1], x10, x11);
// Do final lerp in the Z axis
float finalValue = lerp(samplePosition[2], y00, y01);

An example of a portion of a fragment program in the OpenGL ARB_FRAGMENT_PROGRAM syntax as described above is as follows:

######################################################################
#######
#
# Copyright (c) 2003, Vital Images, Inc.
# All rights reserved worldwide.
#
######################################################################
#######
# 3-region segmentation
#
# Textures:
# 0 - raw grey data: 3D Luminance, liner interp
# 1 - segmentation mask channels: 3D RGBA, linear Interp
# 3 - Segmentation Region 1 transfer function: 1D, RGBA, linear
Interp
# 4 - Segmentation Region 2 transfer function: 1D, RGBA, linear
Interp
# 5 - Segmentation Region 0 transfer function: 1D, RGBA, linear
Interp
######################################################################
#######
# Computed by the VPU
ATTRIB greyTexcoord = fragment.texcoord[0];
ATTRIB segMaskTexcoord = fragment.texcoord[1];
TEMP grey;
TEMP segWts;
TEMP rgba0;
TEMP rgba1;
TEMP rgba2;
# perform trilinear interpolation
TEX grey, greyTexcoord, texture[0], 3D;
TEX segWts, segMaskTexcoord, texture[1], 3D;
# Use grey value as index into RGBA tables for each transfer function
TEX rgba0, grey, texture[3], 1D;
TEX rgba1, grey, texture[4], 1D;
TEX rgba2, grey, texture[5], 1D;
# Combine RGBA values weighted by segmentation region contributions
MUL rgba0, segWts.x, rgba0
MAD rgba0, setWts.y, rgba1, rgba0;
MAD_SAT result.color, segWts.z, rgba2, rgba0

The fragment program resides in the video card memory, but is supplied by the application. The volumetric data may be sent, at 604, in various forms. In one example, the volumetric data is sent as 8-bit unsigned values. In another example, the volumetric data is sent as 16-bit unsigned values. The segmentation mask can be sent at 612 in various forms. In one example, the format is RGBA2, which is an 8-bit format where each of the Red, Blue, Green, and Alpha components use two bits. In another example, the format is RGBA4, a 16-bit format where each of the color components uses 4 bits. In a third example, the format is a compressed texture format that uses one bit per color component. In a fourth example, the format at 612 depends on the number of segmentation regions that are present in predefined subregions of the volume. If only one segmentation region is present in the subregion, it is not necessary to associate segmentation mask volume for the voxels in that subregion because all samples will belong to the same segmentation region.

FIG. 7 is a schematic illustration of one conceptualization of volume rendering using ray-casting (although other volume rendering techniques could also be used). This example of volume rendering uses a rectilinear 3D array of voxels 700, acquired by the imaging device 102, to produce an image on a two dimensional screen 702 (such as the display screen 202) comprising a 2D array of pixels. The 2D image displayed on the screen 702 is as viewed by a user located at a virtual “eye” position 704. As illustrated in FIG. 7, the 3D voxel array 700 may assume an arbitrary position, scale, and rotation with respect to the 2D screen 702 and the virtual eye position 704 (which can be located either outside the voxel array 700, as illustrated, or alternatively located inside the voxel array 700).

This conceptualization of volume rendering uses various rays 705. Each ray 705 is drawn from the virtual eye position 704 through the center of each pixel (e.g., center of a pixel 703) on the screen 702. The rays 705 extend toward the voxel array 700. Some of the rays 705 pass through the voxel array 700. Each voxel through which a ray 705 passes makes a contribution toward the visual characteristics of the pixel 703 corresponding to that particular ray 705. This use of rays 705 is generally known as ray-casting. However, this is only one approach to volume rendering. The present systems and methods are also applicable to other rendering approaches. An example of another such rendering approach used by the present systems and methods includes object-order rendering using texture compositing.

FIG. 7 also illustrates sample points 706 taken along a ray at various locations within the voxel array 700. A fragment program is executed at each sample point 706 on a particular ray 705. The fragment program produces an RGBA (Red—Green—Blue—Opacity) output vector (also referred to as a fragment result) at each sample point 706. These RGBA output vectors for each ray 705 are combined and stored in association with the corresponding pixel 703 through which that particular ray 705 passes. This stored combined RGBA value for the ray 705 determines what is displayed at the corresponding pixel 703. This process is repeated for the sample points 706 on the other rays 705, which intersect the other pixels 703 on the screen 702. In the aggregate, these displayed pixels 703 form a 2D visualization of the imaging data. The conceptual illustration provided in FIG. 7 is only one example of rendering a 2D image of volumetric data on a display 702. The rendered image may be depicted in many forms, including a perspective image, an orthographic image, or the like. The present systems and methods are also applicable to 3D displays as well. One example of such a 3D display is a holographic display. Other examples include formats such as film.

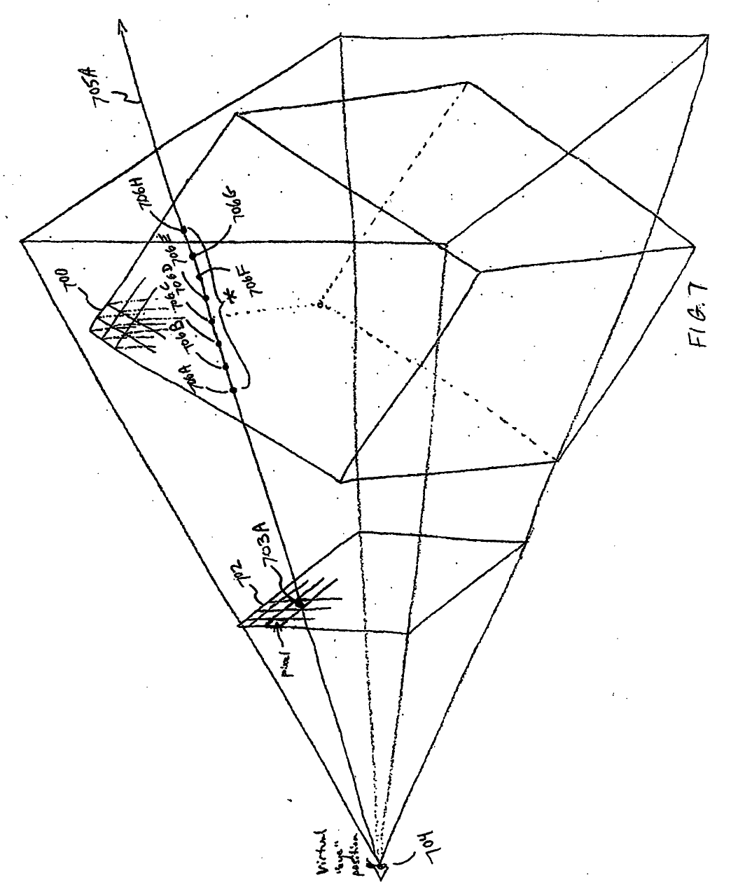

FIG. 8 is a further schematic illustration of the volume rendering conceptualization of FIG. 7, but illustrating at a higher magnification a small portion of the ray 705A as it passes through a neighborhood 800 of eight neighboring voxels (that are defined by their centerpoints 802A-H). These points 802A-H form a box of points on a 3D grid having a particular orientation in 3D space. In this illustrative example, two sample points 706D and 706E, fall within the cubic neighborhood box 800 that is defined by the points 802A-H. There may be a greater or lesser number of sample points 706 that fall within a particular neighborhood box 800. There may also be a greater or lesser number of rays 705 that fall within a particular neighborhood box 800. Thus, FIG. 8 illustrates merely illustrates one example that is useful for providing conceptual clarity.

Each voxel point 802 includes an intensity value (also referred to as a gray value) that defines the intensity of that voxel point 802. Each voxel point 802 also includes a segmentation mask vector that defines which one of the mutually exclusive segmentation regions to which that particular voxel point 802 belongs. Because the sample points 706 do not necessarily coincide with the voxel points 802, the fragment program is used to calculate (e.g., using trilinear interpolation) the intensity (i.e., gray level) contribution of each of the voxel points 802 in the neighborhood box 800 to a particular sample point 706, such as the sample point 706D. For example, a sample point 706 that is located closer to one corner of the neighborhood box 800 will receive a greater intensity contribution from that nearby corner's voxel point 802 than from more distant voxel points 802 in the neighborhood box 800. The programmable video card graphics pipeline also combines the resulting sample point 706 fragment results on a particular ray 705. This produces an aggregate RGBA value for the pixel corresponding to that particular ray 705.

Although, in FIG. 8, each voxel point 802 belongs to only one segmentation region, neighboring voxel points 802 in the same neighborhood box 800 may belong to different segmentation regions. This will be true, for example, for a neighborhood box 800 that lies on a boundary between different segmentation regions. Therefore, the resulting sample points 706 that fall within such a neighborhood box 800 on a boundary will be capable of partially belonging to more than one segmentation region. The extent to which a particular sample point 706 belongs to the different segmentation regions is described by a vector of segmentation “weights” that are computed (e.g., by trilinear interpolation) from the segmentation mask vector of the voxel points 802 in the neighborhood box 800 in which the sample point 706 falls. For example, a sample point 706 that is located closer to one corner of the neighborhood box 800 will receive a greater segmentation region contribution from that nearby voxel point 802 than from more distant voxel points 802 in the neighborhood box 800. The fragment program executes for each sample point (e.g., 706D and 706E) along a ray (e.g., 705A) and the segmentation masks in the neighborhood of each sample point 706 are trilinearly interpolated to obtain a segmentation weight vector corresponding to that sample point. (As discussed above, the programmable video card graphics pipeline also combines the resulting sample point 706 fragment results on a particular ray 705. This produces an aggregate RGBA value for the pixel corresponding to that particular ray 705.) Thus, in the above example, for a given sample point 706, trilinear interpolation is performed both on: (1) the intensity values of the nearest neighbor voxel points 802 defining the neighborhood box 800 containing that sample point 706; and, (2) on the voxel segmentation mask information of the same nearest neighbor voxel points 802 defining the neighborhood box 800. In addition to trilinear interpolation, other examples of substitutable interpolations include cubic spline interpolation, or other interpolations. In two-dimensional applications a bilinear interpolation or other interpolations could be used.

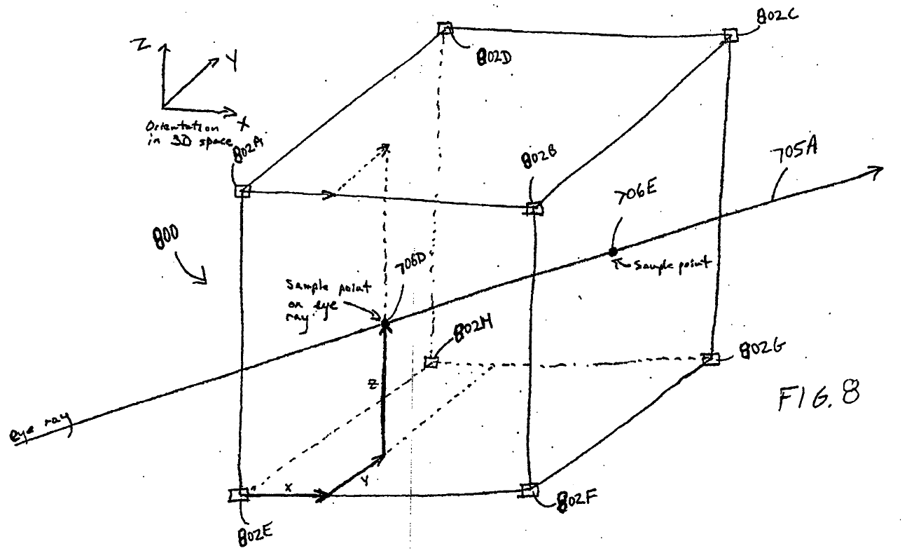

FIG. 9 is an illustration of one example of using transfer functions to overlay different visual characteristics to voxel intensity data that is associated with different segmentation regions. For conceptual clarity, FIG. 9 illustrates a case having three different segmentation regions: Segmentation Region 0, Segmentation Region 1, and Segmentation Region 2. However, the present systems and methods can render an arbitrary number of segmentation regions, such as by extending the segmentation mask to be stored in additional 4-vector storage in the video card memory 320. The exact number of segmentation regions will vary depending on the particular application.

As an illustrative example, suppose that Segmentation Region 0 includes segmented voxel data that was deemed “uninteresting” by a segmentation algorithm or manually. Also suppose that Segmentation Region 1 includes segmented voxel data that a segmentation algorithm deemed to be associated with vessels in the imaged structure, which are of particular interest to a user. This might be the case, for example, in an application in which a cardiologist is interested in assessing the degree of stenosis in a coronary blood vessel, for example. (Alternatively, segmentation may be used for segregating voxel data associated with another tubular structure, such as a colon, or another organ, such as a liver, etc.) Continuing with the coronary vessels illustrative example, suppose that Segmentation Region 2 includes segmented voxel data that a segmentation algorithm deemed to be associated with a heart (other than blood vessels associated with the heart, which would be in Segmentation Region 1).

FIG. 9 includes one transfer function 900 corresponding to each segmentation region. In the present example, which includes three segmentation regions, there are three transfer functions. For example, transfer function 900A is associated with the “uninteresting data” of Segmentation Region 0. The transfer function 900B is associated with the “blood vessel” data of Segmentation Region 1. The transfer function 900C is associated with the “heart” data of Segmentation Region 2.

In one example, each transfer function 900 is represented by a mathematical function that calculates visualization values for a given input intensity value and/or other values (e.g., gradient, magnitude, etc.). As an example, such a function may be implemented as additional instructions within the same fragment program as described above.

In this example, each transfer function 900 includes an array of N visualization values. The number N of visualization values in each array typically derives from the resolution of the voxel intensity values of the acquired imaging dataset. In one example, each voxel intensity value is represented as a 16-bit unsigned integer. This yields N=2¹⁶=65,536 possible different intensity levels for each voxel. Therefore, in this example, each transfer function array includes 65,536 elements. Another example provides that, for the same 65,536 possible different intensity levels for each voxel each transfer function array includes only 2¹¹=2,048 different entries. Thus, in this example, the transfer function table is compressed; one transfer function table entry can correspond to multiple intensity values.

Another example uses 2D transfer function tables, as in pre-integrated volume rendering. In such a 2D table, one axis represents the sampled point intensity value going into a thin slab of volumetric data along a ray, and the other axis represents the sampled point intensity value coming out of the thin volumetric slab. Another example uses N-dimensional transfer function tables that are indexed by various multiple values including intensity, gradient magnitude, etc.

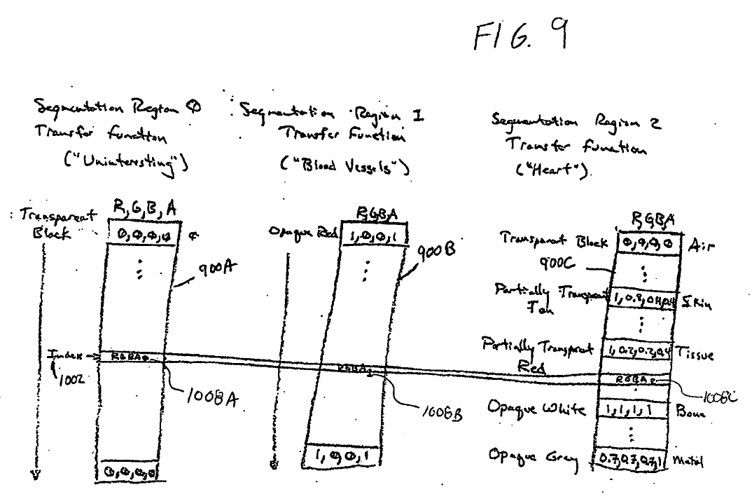

One technique represents the visualization values as RGBA values (each RGBA value is itself a vector that includes 4 elements, each element describing a respective one of a Red color level, a Green color level, a Blue color level, and an Opacity (or, its inverse, Transparency) level). A particular voxel's intensity value is used as an index 1002 (described further with respect to FIG. 10) into each array of the transfer functions 900A-C. In one example, as discussed above, for each sample point 706 to which a transfer function 900 is applied, the intensity value (used as the index 1002) is the result of a trilinear interpolation of the contribution of the eight nearest neighbor voxel points 802 defining the neighborhood box 800 in which that sample point 706 resides.

In the illustrative example of FIG. 9, the transfer function 900A maps every intensity level of the “uninteresting” data of Segmentation Region 0 to a transparent RGBA value. A transparent RGBA value is specified by the RGBA vector (0, 0, 0, 0). Since every intensity level is being mapped to transparent for Segmentation Region 0, each element in the array of the transfer function 900A contains the transparent RGBA vector (0, 0, 0, 0).

In this same example, the transfer function 900B maps every intensity level of the “blood vessel” data of Segmentation Region 1 to an opaque red RGBA value. An opaque red RGBA value is specified by the RGBA vector (1, 0, 0, 1). Since every intensity level is being mapped to opaque red for Segmentation Region 1, each element in the array of the transfer function 900B contains the opaque red RGBA vector (1, 0, 0, 1).

In this same example, the transfer function 900C maps various different intensity levels of the “heart” data of Segmentation Region 2 to various different RGBA values. In this illustrative example (which corresponds to a CT imaging example), low intensity levels corresponding to low density air (such as contained in voxels corresponding to the nearby lungs) are mapped to a transparent RGBA value of (0, 0, 0, 0). Similarly, in this example, the slightly higher intensity levels of slightly higher density skin are mapped to a partially transparent tan RGBA value of (1, 0.8, 0.4, 0.4). The even slightly higher intensity levels of even slightly higher density tissue are mapped to a partially transparent red RGBA value of (1, 0.2, 0.2, 0.4). The even higher intensity levels of even higher density bone are mapped to an opaque white RGBA value of (1, 1, 1, 1). An ultra high intensity level of ultra high density metal (e.g., an implanted pacemaker lead, etc.) is mapped to an opaque gray RGBA value of (0.7, 0.7, 0.7, 1). In this fashion, the segmented heart data will have different visual characteristics for different structures within the segmented heart data.

FIG. 10 is a schematic diagram illustrating conceptually how, for each sample point 706, the fragment program uses the interpolated voxel intensity value 1002, the interpolated segmentation weight vector 1004, and the transfer functions 900. FIG. 10 illustrates an example having three segmentation regions, such as discussed above with respect to FIG. 9. However, a different number of segmentation regions may also be used. In the example of FIG. 10, the volume data 700 is used to generate the interpolated voxel intensity value 1002 corresponding to the particular sample point 706. Corresponding to the volumetric intensity data 700 is segmentation data 1006. The segmentation data 1006 includes a corresponding segmentation mask vector for each voxel in the volume data 700. Each voxel's segmentation vector defines to which one of the segmentation regions that particular voxel was assigned. In the example of FIG. 10, the segmentation data 1006 is used to generate the interpolated segmentation weight vector 1004 corresponding to the particular sample point 706. The segmentation weight vector 1004 includes weight elements 1007A-C corresponding to the Segmentation Region 0, Segmentation Region 1, and Segmentation Region 2, respectively.

In FIG. 10, the interpolated voxel intensity value 1002 is used as an index into each of the transfer functions 900A, 900B, and 900C to retrieve a respective visualization value 1008A-C (in this case, an RGBA value) from each of the respective transfer functions 900A-C. Each of the retrieved RGBA visualization values 1008A-C is multiplied by its respective segmentation weight 1007 to form an addend. These addends are summed to output a composite RGBA visualization value 1010 (also referred to as a “fragment result”) for the particular sample point 706. In some examples, the RGBA output value 1010 is also modulated by local lighting calculations or other calculations. The RGBA output value 1010 is composited into the frame buffer 410 by the blending unit 408. This process is repeated for each sample point 706 on a particular ray 705. At the end of this loop, the pixel 703 corresponding to that particular ray 705 contains an aggregate visualization value suitable for display or other output.

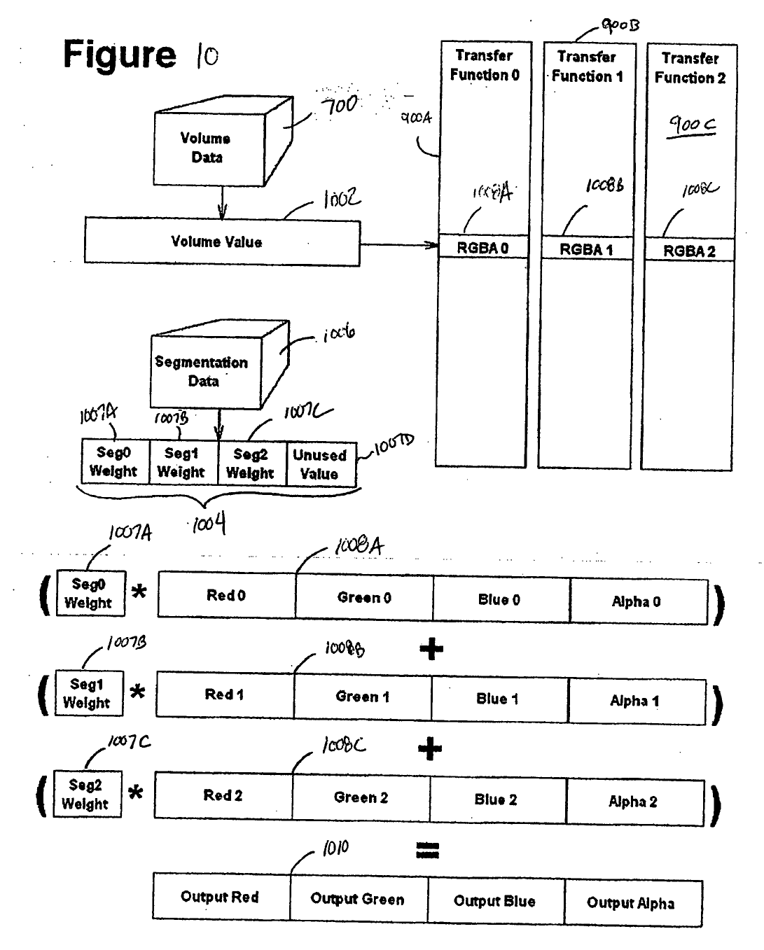

FIG. 11 is a schematic diagram illustrating conceptually one example of various data structures associated with an exemplary fragment shading segmented volume rendering process. The exemplary data format 1100 illustrates storing voxel intensity data as a 16 bit unsigned integer. In this example, an “empty” voxel 1000A (i.e., intensity=0) is characterized by all bits being zeros. A “full” voxel 1000B (i.e., intensity=full scale) is characterized by all bits being ones. A exemplary voxel 1000C that is “37% full” is characterized by a bit string of “0101 1110 1011 1000.”

The exemplary data format 1102 illustrates storing a segmentation mask vector having four channels of 4-bit segmentation data, such as where it is convenient to do so. A voxel in Segmentation Region 0 has only the first element (i.e., Segmentation Region 0 weight 1007A) asserted, yielding a segmentation vector 1004A that is characterized by a bit string of “1111 0000 0000 0000.” A voxel in Segmentation Region 1 has only the second element (i.e., Segmentation Region 1 weight 1007B) asserted, yielding a segmentation vector that is characterized by a bit string of “0000 1111 0000 0000.” A voxel in Segmentation Region 2 has only the third element (i.e., Segmentation Region 2 weight 1007C) asserted, yielding a segmentation vector 1004C that is characterized by a bit string of “0000 0000 1111 0000.” Since there are only three segmentation regions, in this example, the fourth field (i.e., 1007D) of the segmentation vector 1004 is not used.

The exemplary data format 1104 illustrates storing each RGBA visualization data value 1008 as 32 bits. For example, a completely white opaque RGBA value 1008D is stored as the bit string “1111 1111 1111 1111 1111 1111 1111 1111.” A completely red opaque RGBA value 1008E is stored as the bit string “1111 1111 0000 0000 0000 0000 1111 1111.” A completely invisible RGBA value 1008F is stored as the bit string “0000 0000 0000 0000 0000 0000 0000 0000.” A semi-transparent pink RGBA value 1008G is stored as the bit string “1111 1001 1010 1001 1100 1011 1000 1100.”

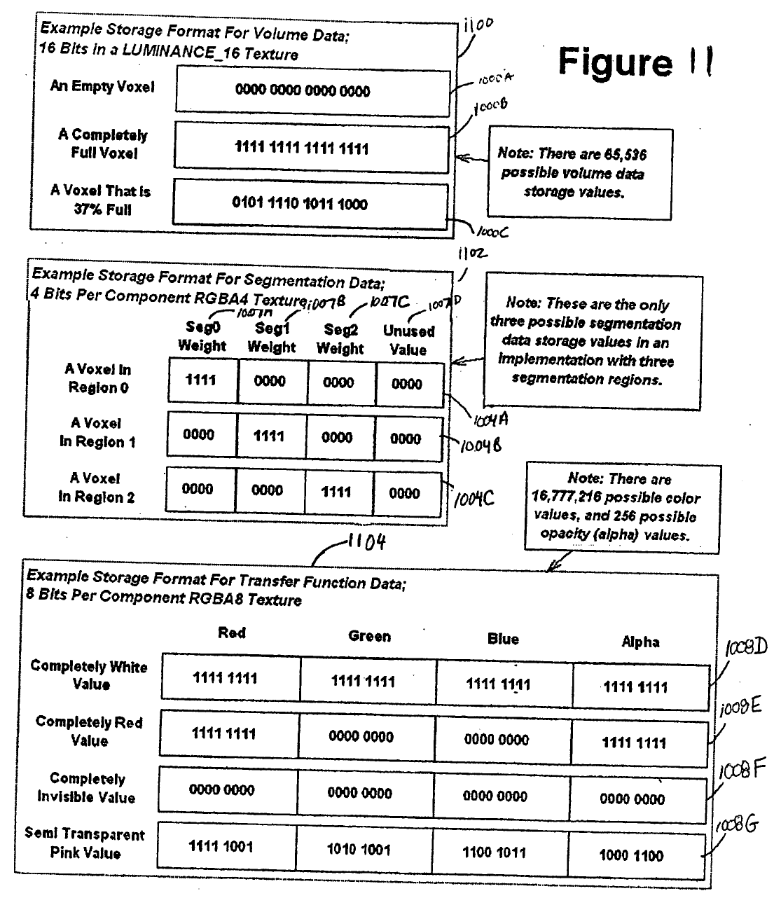

FIGS. 12-14 are schematic diagrams illustrating conceptually how segmentation weights 1007 are derived. FIG. 12 is a schematic diagram of a neighborhood block 800 comprising voxel points 802A-H and a sample point 706 contained within that neighborhood block 800. In this example, voxel points 802A, 802E, 802H, and 802D are all in Segmentation Region 0. Voxel points 802B and 802C are both in Segmentation Region 1. Voxel points 802F and 802G are both in Segmentation Region 2.

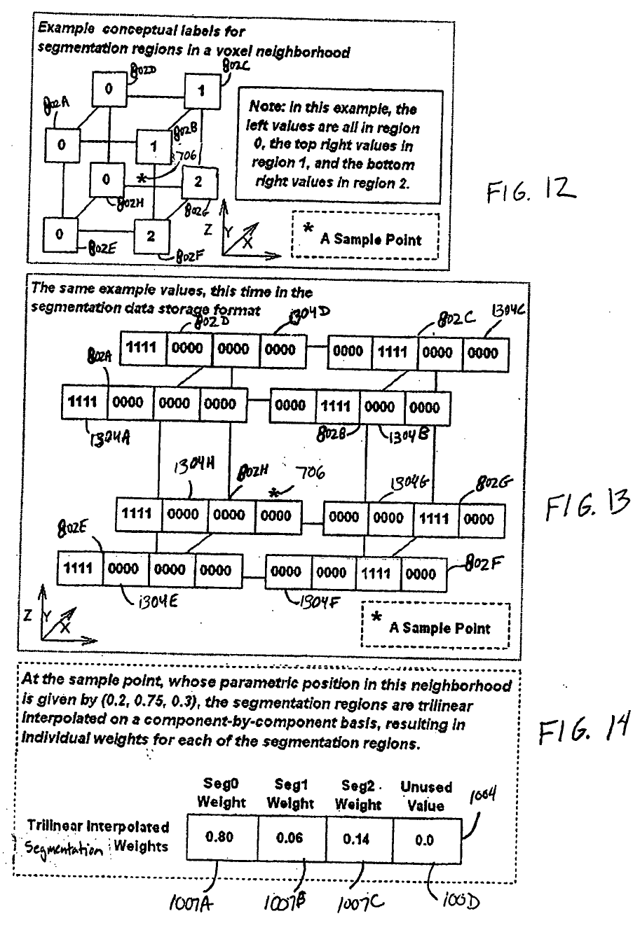

FIG. 13 is a schematic diagram, corresponding to the same neighborhood block 800 of FIG. 8, but with the voxel points 802 represented by their respective segmentation mask values composed of four channels of 4-bit unsigned integer data values. The 16 bit unsigned integer data values represent 4-element segmentation vectors 1304 indicating to which segmentation region that particular voxel point 802 belongs. Each voxel point 802 belongs to at most one segmentation region.

FIG. 14 is a schematic diagram illustrating the result of a trilinear interpolation (on a component-by-component basis) on a sample point 706 having parametric (x, y, z) coordinates of, for instance, (20.2, 186.75, 40.3). The neighborhood block 800 is selected as the eight voxels surrounding the coordinates (20, 186, 40). Then, the fractional components within the neighborhood block 800 are used—in this example, (0.2, 0.75, 0.3). For these sample point 706 coordinates, the resulting interpolated segmentation weight vector 1004 is (0.80, 0.06, 0.14, and 0.0).

Although the examples discussed above have focused on medical imaging, the present systems and methods will find many other applications. For example, such systems and methods could also be implemented in a video game or other system that includes rendering of volumetric data, such as that describing smoke, clouds, or other volumetric phenomenon.

Among other things, the present systems and methods are both storage and computationally efficient on commodity video cards and graphics programming APIs. These systems and methods allow for lower cost volumetric data image rendering systems while providing higher resolution images.

Among other things, the present systems and methods leverage the computational efficiency of commodity programmable video cards to determine accurately subsampled partial contribution weights of multiple segmented data regions to allow correct per-fragment contributions of segment-specific characteristics such as color and opacity suitable for applications including volume rendering.

It is to be understood that the above description is intended to be illustrative, and not restrictive. For example, the above-described examples (and/or aspects thereof) may be used in combination with each other. Many other examples will be apparent to those of skill in the art upon reviewing the above description. The scope of the invention should, therefore, be determined with reference to the appended claims, along with the full scope of equivalents to which such claims are entitled. Functions described or claimed in this document may be performed by any means, including, but not limited to, the particular structures described in the specification of this document. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects.

1. A computer implemented method comprising:
obtaining three dimensional (3D) volumetric data comprising voxels, each voxel comprising a corresponding intensity value;
segmenting the volumetric data to create a segmentation mask volume having one or more segmentation regions, wherein each voxel is assigned to only one of the segmentation regions by a segmentation mask vector;
obtaining a transfer function for each segmentation region, each transfer function including visualization values;
selecting sample points at various locations within the volumetric data;
computing an interpolated intensity value for each sample point by interpolating intensity values of voxels near a particular one of the sample points;
using the interpolated intensity values for each sample point to obtain for each sample point visualization values from respective transfer functions, the obtaining including using the interpolated intensity value for the particular one of the sample points as an index into the transfer functions;
computing interpolated segmentation mask weights for each sample point by interpolating segmentation mask vector values of voxels near the particular one of the sample points;
multiplying, for each sample point, respective visualization values by corresponding segmentation weights to form addends;
summing the addends to obtain a fragment result that includes a composite visualization value;
combining the fragment results into pixel values; and
wherein the selecting sample points, the computing the interpolated intensity value for each sample point, the using the interpolated intensity values for each sample point for obtaining for each sample point visualization values, the computing interpolated segmentation mask weights, the multiplying respective visualization values, the summing the addends, and the combining the fragment results into pixel values, are performed using a graphics processing unit (GPU) of a programmable video card.

2. The method of claim 1, wherein the combining the fragments into pixel values includes using at least one of compositing and ray-casting.

3. The method of claim 1, wherein the volumetric data is acquired using a medical imaging device.

4. The method of claim 1, wherein the volumetric data is produced by a video game system.

5. A computer implemented method comprising:
receiving, at a programmable video card, three dimensional (3D) volumetric data including intensity values, a segmentation mask volume data classifying the volumetric data into segmentation regions using segmentation mask vectors, and at least one rendering program that calculates, for each of the segmentation regions, at least one visualization value that is specific to that particular segmentation region; and
processing the received data using the programmable video card, the processing including computing sample points at locations within the volumetric data, and for each sample point:
obtaining a vector of visualization values, each visualization value in the vector corresponding to one of the segmentation regions.
interpolating neighboring segmentation mask vectors to obtain a segmentation weight vector;
multiplying respective visualization values by corresponding segmentation weights to obtain addends; and
summing the addends to obtain a fragment value; and
combining fragment values into pixel values.

6. The method of claim 5, wherein the visualization values and the fragment values are RGBA values representing color and opacity.

7. The method of claim 5, wherein the combining fragments includes performing at least one of compositing and ray-casting.

8. The method of claim 5, wherein the volumetric data is acquired by a medical imaging device.

9. The method of claim 5, wherein the volumetric data is produced by a video game system.

10. A system comprising:
a processor;
a programmable video card, operatively coupled to the processor, the video card including video output port;
software operable on the processor to:
obtain volumetric data comprising voxels having respective intensity values;
create a segmentation mask vector for each voxel, classifying the voxel into a particular one of different segmentation regions;
encode, for each segmentation region, information that includes at least one of at least one visualization value and at least one rendering program;
send the volumetric data, the segmentation mask vectors, and the encoded information to the programmable video card; and
a fragment program executable on the video card to process sample points at locations within the volumetric data, and for each sample point:
calculate a vector of visualization values, each visualization value in the vector corresponding to one of the segmentation regions.
interpolate neighboring segmentation mask vectors to obtain a segmentation weight vector;
multiply respective visualization values by corresponding segmentation weights to obtain addends; and
sum the addends to obtain a fragment result; and
combining fragment results into pixel values.

11. The system of claim 10, wherein the visualization values include RGBA values.

12. The system of claim 10, wherein the combining fragment values into pixel values includes performing ray-casting.

13. The system of claim 10, wherein the video card is an OpenGL compliant computer video card.

14. The system of claim 10, wherein the video card is a Direct3D compliant video card.

15. The system of claim 10, wherein the volumetric data is produced by a medical imaging device.

16. The system of claim 15, wherein the medical imaging device is a CT scanner.

17. The system of claim 10, wherein the volumetric data is produced by a video game system.

18. A programmable video card comprising:
a graphics processing unit;
a memory;
an graphics port connection;
a video output; and
software operable to instruct the video card to:
receive volumetric data comprising voxels having respective intensity values;
receive a segmentation mask vector for each voxel, the segmentation mask classifying the voxel into a particular one of different segmentation regions;
receive separate encoding of visualization information for each different segmentation region; and
define sample points at locations within the volumetric data, and for each sample point, the software further operable to instruct the video card to:
calculate a vector of visualization values, each visualization value in the vector corresponding to one of the segmentation regions;
interpolate neighboring segmentation mask vectors to obtain a segmentation weight vector;
multiply respective visualization values, in the vector of visualization values, by corresponding segmentation weights to obtain addends; and
sum the addends to obtain a fragment result; and
combine the fragment results into pixel values.

19. A computer readable medium including instructions that, when executed on a properly configured device, perform a method comprising:
obtaining three dimensional (3D) volumetric data comprising voxels, each voxel comprising a corresponding intensity value;
segmenting the volumetric data to create a segmentation mask volume having one or more segmentation regions, wherein each voxel is assigned to only one of the segmentation regions by a segmentation mask vector;
obtaining a transfer function for each segmentation region, each transfer function including visualization values;
selecting sample points at various locations within the volumetric data;
computing an interpolated intensity value for each sample point by interpolating intensity values of voxels near a particular one of the sample points;
using the interpolated intensity values for each sample point to obtain for each sample point visualization values from respective transfer functions, the obtaining including using the interpolated intensity value for the particular one of the sample points as an index into the transfer functions;
computing interpolated segmentation mask weights for each sample point by interpolating segmentation mask vector values of voxels near the particular one of the sample points;
multiplying, for each sample point, respective visualization values by corresponding segmentation weights to form addends;
summing the addends to obtain a fragment result that includes a composite visualization value;
combining the fragment results into pixel values; and
wherein the selecting sample points, the computing the interpolated intensity value for each sample point, the using the interpolated intensity values for each sample point for obtaining for each sample point visualization values, the computing interpolated segmentation mask weights, the multiplying respective visualization values, the summing the addends, and the combining the fragment results into pixel values, are performed using a graphics processing unit (GPU) of a programmable video card.
